(19) United States
(12) Patent Application Publication
(10) Pub. No.: US 2011/0177467 A1
(43) Pub. Date: Jul. 21, 2011

(54) ADAPTABLE DEVICE FOR DETECTING AND TREATING DENTAL PATHOLOGIES

(75) Inventor: Naim KARAZIVAN, Repentigny (CA)

(73) Assignee: DENTSPLY CANADA LTD., Woodbridge (CA)

(21) Appl. No.: 13/009,480

(22) Filed: Jan. 19, 2011

Related U.S. Application Data
(62) Division of application No. 11/871,907, filed on Oct. 12, 2007, now abandoned.
(60) Provisional application No. 60/829,248, filed on Oct. 12, 2006.

(51) Int. Cl. A61B 6/14

(52) U.S. Cl. 433/29

(57) ABSTRACT

There is disclosed a tartar removal tip having a ceramic distal portion and a metal proximal portion. An optical dental pathology detection system has a housing that mates with a handle of a tartar removal instrument. A replacement tip for a tartar removal device has integrated optical components and a coupler for coupling to a dental pathology detection system without interacting or changing a handle of an instrument to which the replacement tip connects.

### FIELD OF THE INVENTION

This invention relates to the field of dental devices, and more specifically to devices for detection of pathologies and dental treatment.

### BACKGROUND OF THE INVENTION

Optical detection apparatuses have been described for use in detection of dental pathologies such as caries, tartar, fractures and the like. Such detection systems have also been described in combination with existing dental tools, such as tartar removal instruments. In commonly assigned U.S. patent publication 2004/0106081, the specification of which is hereby incorporated by reference, a tartar removal instrument is provided with a modified handle and tip equipped with an optical light guide to allow a user to detect tartar at the working end of the tartar removal instrument. However, these tools lack flexibility and adaptability with regard to their use for multiple applications such as detection of pathologies and treatment of the pathologies. Accordingly there is a need for improved dental tools.

In powered scaling or tartar removal instruments, the tip is normally part of an insert. The tip is a replaceable part that is subject to wear and replacement after such wear.

### SUMMARY OF THE INVENTION

In a broad embodiment of the invention, there is provided a dental instrument tip that has a proximal portion made of a material able to withstand flexion and/or vibration and a distal portion made of a more brittle material having a better hardness at the working end.

In one aspect, a tartar removal tip has a metal proximal portion and a ceramic distal portion. In some embodiments, the tip is equipped with one or more optical fibers that extend along the proximal portion into the distal portion for the purposes of emitting and/or collecting light as part of a tartar or other dental pathology detection system.

In a broad embodiment of the invention there is provided an insert comprising optical means for detection of dental pathologies, said insert being adapted to be placed on a functional part of a dental instrument without requiring modification of the dental instrument to be equipped with the optical detection capability.

In one aspect of the invention, the insert has a suitable coupling for the optical detection system that is on the tip insert such that it does not change or affect the handle portion of the instrument. In another aspect of the invention, there is provided a sheath for a dental instrument tip that fits over an existing dental instrument tip and includes optical components for an optical dental pathology detection system.

In an aspect of the invention the insert is adapted to be placed at a tip of a dental cleaning instrument and the optical components can be protected by wear resistant material.

In another aspect of the invention, a sheath for a tip of a dental instrument

### BRIEF DESCRIPTION OF THE DRAWINGS

Further features and advantages of the present invention will become apparent from the following detailed description, taken in combination with the appended drawings, in which:

FIG. 1a illustrates a sectional side view of a tartar removal tip including an optical fiber and a translucent ceramic tip covering the end of the fiber;

FIG. 1b illustrates a sectional side view of a tartar removal tip similar to FIG. 1a in which the ceramic tip is opaque and has an aperture for light transmission;

FIG. 2 illustrates a sectional side view of a tartar removal tip and sheath, the sheath being adapted to support an optical fiber end for optical detection of tartar;

FIG. 3 illustrates a removable, sterilizable, tartar removal tip insert adapted to provide a coupler for the optical detection system without having a handle modification;

FIG. 4 is a sectional side view of a self-contained, sterilizable tartar detection system that fits over and around a handle of a powered tartar removal instrument and is connected to a replaceable tip of the instrument. the tip having optical components for the detection of tartar;

FIG. 5 is a sectional view of the instrument of FIG. 4 about plane 5-5;

FIG. 6 is a section view similar to that of FIG. 5, in an embodiment similar to FIGS. 7 and 8;

FIG. 7 is a perspective view of a tartar removal instrument having a fit over optical tartar detection system that form part of an enlarged handle of the instrument;

FIG. 8 is a bottom view of the fit over system housing separate from the instrument;

FIG. 9 is a side sectional view of an embodiment in which a self-contained, sterilizable tartar detection system is integrated as part of an insert tip by providing an extension of the insert shaft to receive a detection system housing on the shaft.

### DETAILED DESCRIPTION OF THE INVENTION

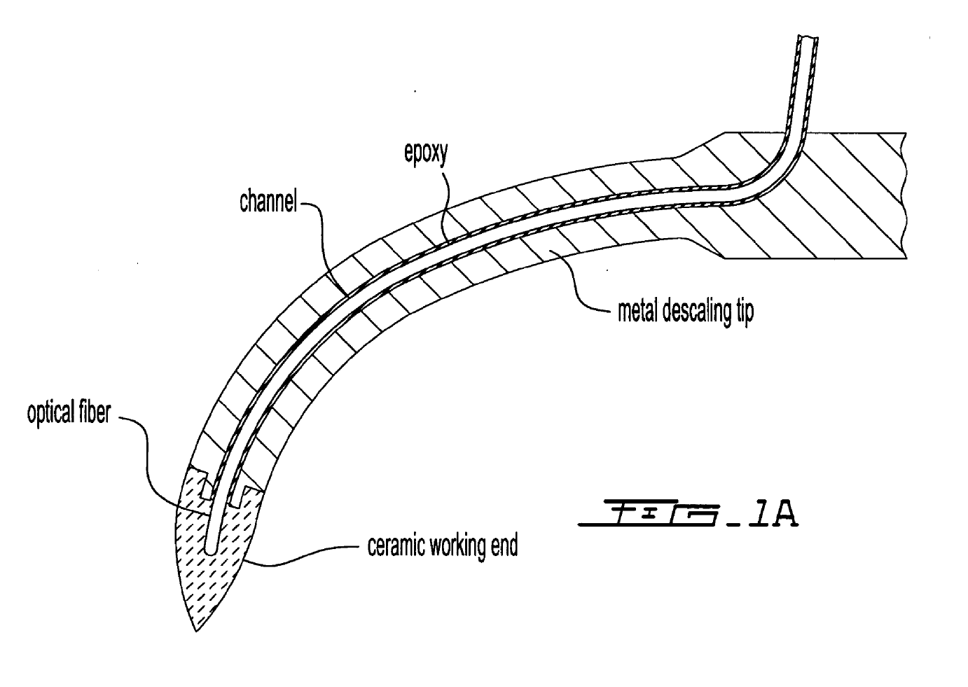

According to a first embodiment shown in FIG. 1a and 1b, a tartar removal tip, also known as a scaling tip, has a hybrid construction with a first proximal part made of metal and a second distal part made of ceramic. The metal provides the ability to flex under the conditions of being subjected to pressure by the user acting on the tooth, and under conditions of high intensity vibrations originating from the powered scaling instrument. The ceramic tip has a hardness that allows it to be more wear resistance than the proximal part. A scaling tip made entirely of conventional ceramic would be prone to breaking under the mechanical stresses that the tip is subjected to.

In the embodiment of FIG. 1a, the ceramic working end is transparent or translucent and the optical fiber terminates centrally inside the ceramic portion. The fiber is made solid with the metal and ceramic portions using epoxy, so that vibrations do not damage the fiber or fibers. In the embodiment of FIG. 1b, the ceramic tip has a channel ending in an aperture for the fiber. While the end of the fiber is recessed within the ceramic tip, and thus protected from wear or damage, light may pass through the aperture. The aperture may be filled with another transparent or translucent material to protect the end of the fiber within the channel.

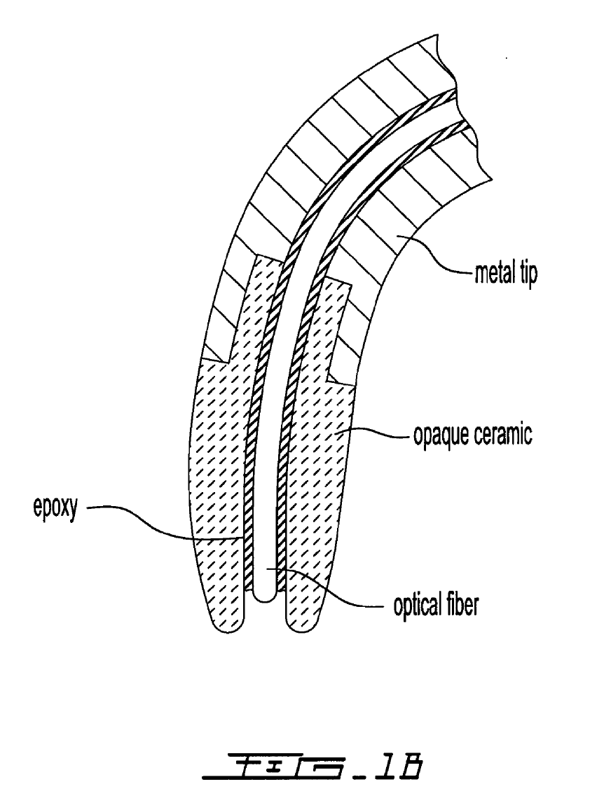

In the embodiment of FIG. 2, the ceramic working end is provide as a sheath to press-fit over a conventional tartar removal tip. In this embodiment, the optical fiber is fed along a side of the sheath and terminates either to a side or centrally within the ceramic working tip.

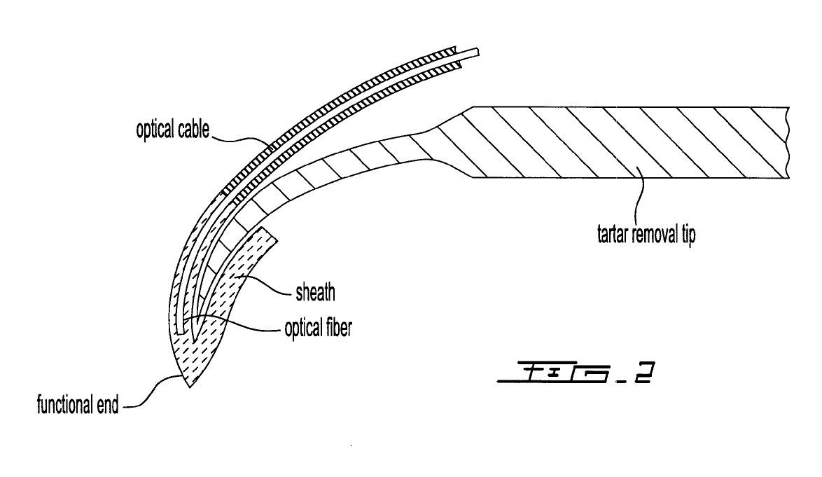

In the embodiment of FIG. 3, the scaler tip is modified to integrate an optical fiber, either as show with a parallel optical fiber support or by using a central channel in the tip to guide the fiber to the end of the tip. The tip is somewhat longer than a conventional replacement tip for the desired model of powered scaler, and a coupler is provided on an extension portion. The coupler may be an optical coupler for coupling the one or more optical fibers to an optical cable leading to the optical tartar (and/or other dental pathology) detection system. Alternatively, a light source and light detector may be provided within the illustrated coupler block, and the coupler may be an electrical coupler. The tartar removal tip is mechanically attached within the scaler instrument and driven to have the desired scaling action at the tip.

There is provided an insert tip comprising optical means for detection of dental pathologies that can be inserted into an existing dental instrument such as a powered sonic or ultrasonic instrument or a rotary instrument for cleaning teeth. The tip can be connected to an optical analyzing means to detect the presence of dental pathologies such as calculus, caries, plaque, blood, dental fractures and the like. The tip of the present invention advantageously enables the user to adapt existing dental cleaning instruments for the detection of dental pathologies. In one embodiment the tip is adapted to perform the function of the instrument (such as cleaning tartar) when placed on the instrument. In one embodiment the tip is advantageously designed to be removably attached (“retrofit” tip) to the device allowing repeated use of the same tip, easy replacement of the tip with another tip or for sterilization purposes. The tip may also be disposable.

Instruments on which the tip can be inserted comprise but are not limited to: Sonic Instruments such as ultrasonic Instruments (piezoelectric e.g. EMS or ACTEON, magnetostricitive e.g. Dentsply Inc.) and hand instruments such as rotary instruments.

The optical components are at least in part comprised within the tip and are adapted to resist wear during normal use of the instrument. In particular, the optical components should be protected from friction and water. Various designs can be used to achieve these goals.

In one embodiment, the tip can be designed such that the light injection port and the detection port are in direct contact with the teeth when in use. These optical components may be surrounded by hard and wear resistant materials. The optical means inside the tip can be uncovered and terminate at the extremity of the probe tip. Having hard materials around these optical means protects the optical means even if the optical means are in direct contact with dental structures. Material such as sapphire, ceramics, tungsten carbide, zirconia, ruby or other very hard material can be used to increase resistance. High hardness epoxy can also be used to attach the optical means to the probe tip.

The optical detection means could be covered and protected from stress with a disposable sleeve. The material used could be plastic, epoxy, Teflon® PTFE or other translucent materials.

In another embodiment, the tip can consist of a wear resistant shell completely surrounding the optical components. Material translucent or transparent to the wavelength(s) used for detection of the pathologies can be used to cover optical components. Examples of such materials include but are not limited to Teflon coated tip, Epoxy coating, plastic.

It will be appreciated that when the optical components are covered with translucent or transparent material, reflection inside this material should be taken into account to adjust detection parameters.

The tip may be partially or completely made with wear resistant materials. For example, the insert tip can be made solely in ceramic or partially made in ceramic. However, because ceramic can be susceptible to fracture, the proportion of ceramic can be adjusted while still being compatible with the function of the instrument. The junction between the insert and the instrument (typically metal) can be realized by laser fusion fixation, pressfit fixation, Epoxy fixation, ceramic coating and other such processes as would be known to one skilled in the art.

Because the tip can be made with wear resistant material it can be shaped with a special morphology to enhance its functionality such as calculus removal efficiency. For example hemispheric shaped tips can be designed for this purpose. The tip of the insert can include domes or grooves to enhance scaling performance. The shape of the insert tip can increase the number of stressing point in contact with the calculus and therefore increase the speed of calculus removal and decrease the damage to tooth surface. This can be more easily made with ceramic because of its high resistance to wear. It is also possible to include material such as diamond that will enhance removal of hard tissue.

Fiber Assembly in the Insert:

Plastic fiber: The fibers in the detection optical means can be plastic fiber optics. Plastic fibers cost less (can be made disposable), are less susceptible to fractures when subjected to vibrations, have thinner core to maximize examination area.

Removable fibers: The fiber can be inserted in the tip such as to be removable.

Retractable fibers: The insert tip can be the cap of the optical detection that protects the fibers which can be retracted when not in use.

In the case of instruments using a bur the optic fibers can be incorporated inside the bur and connections made at the tip of the bur.

In another embodiment, optical detection means (ODM) can be protected by using a reduced power strategy. Thus to reduce wear impact on ODM is to instruct the operator to reduce power of the powered instrument. This will enable the tip to wear slowly and therefore keep the tip and ODM working for a longer period. Stainless steel, like actual marketed tips, could be used with this strategy.

Optical components comprise an optical analyzing means (OAM) which can be made to be removable and be coupled with another unit or insert. The OAM can be positioned on the powered scaler unit and use fiber optics to go along the cable and then connect to the insert. The OAM can be adapted to be fitted on the handle or at the cable end of the powered ultrasonic or sonic instrument and connect with or without fiber optics to the insert. The OAM can be inside the insert's handle (specially for magnetostrictive inserts) the OAM can be made to be sterilizable but preferably be removable from the insert handle before sterilization.

It is possible to think to optically connect the insert to the cable optical connector or directly to optical analyzing means with optical path only (without physical contact). For example focusing lenses could be used to focus light onto fiber optics on the insert. This could reduce the impact of vibrations on physical components.

The optical components may comprise components that are well known in the art such as optic fibers, mirrors, reflectors and the like. The optical assembly is designed such as to resist wear during normal use and to be resistant to multiple sterilizations. In this respect it is desirable to provide friction protection of fiber optics using Teflon® tubing for example. For water protection Silicone or the like can be used.

The optic fibers can be cleaved and otherwise processed as needed before insertion into the tip insert. In one embodiment the fibers can be fixed inside the insert with Epoxy having a high hardness and heat resistance.

To protect the fibers from friction stress inside the insert a buffer (made of rubber, plastic, gel, silicone, Teflon, or polished metal . . . ) can be inserted between the fibers and the insert rigid structure. The surface in contact with fiber optics can be treated to reduce friction (sand polishing, chemically treated (acids), metal deposit using electricity . . . )

To verify the smoothness of the insert tip, to determine if the probe tip is not altered and dangerous for the tooth structure a hard material can be used to scrub on it the insert tip and determine if the tip can be used. If deep scratches (or resistance is noted during or after scratching) it may mean that the probe tip has been broken and therefore should not be used to prevent damage of tooth surface.

The various parts of the device are preferably sterilizable. The optical cable or a part of the cable (more particularly the portion of the optical cable next to the insert) can be made to be detachable from OAM. Such as to enable sterilization.

Tip connection detection can be provided to ensure proper connections.

The insert tip can also include a pocket measurement means to prevent changing instrument in order to proceed with periodontal pocket measurement.

The detection technology can be used for caries detection. The sonic or ultrasonic powered instruments can be used for caries removal or preparation of the tooth structure. The detection can also be adapted to be integrated inside a rotary drilling insert for high speed or slow speed. For caries detection the device can be separated from the working tip and be attached only on the handle.

In one embodiment the detection and the “working” tip are separated where more particularly the detection tip can be retractable by using for example a flexible material containing the optical component. The operator can accordingly deploy or retract the detection tip during the session where the working tip is used. It is then more convenient than having to switch to an independent detection instrument.

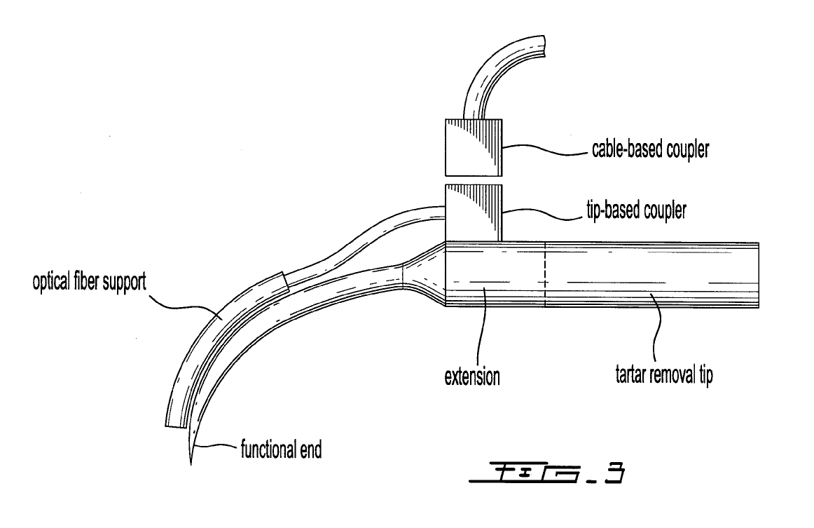

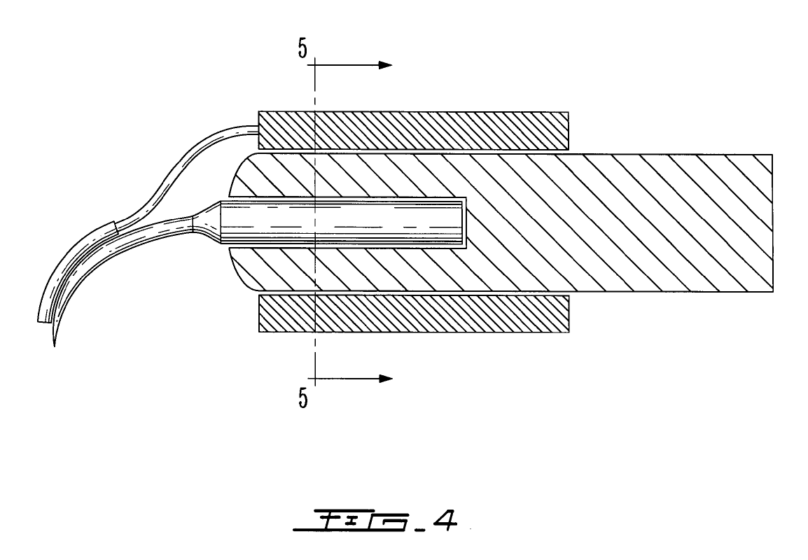

In the embodiment of FIGS. 4 and 5, the optical tartar detection system has a self-contained housing that conforms to the shape of the tartar removal instrument. While the handle of the instrument is enlarged by the addition of the detection system, the combination can still be hand-held and manipulated for use. The optical fiber connection between the tip and the detection system housing can be permanent and sealed, but is preferably detachable. The optical detection system thus includes the tartar removal tip and can be sterilized.

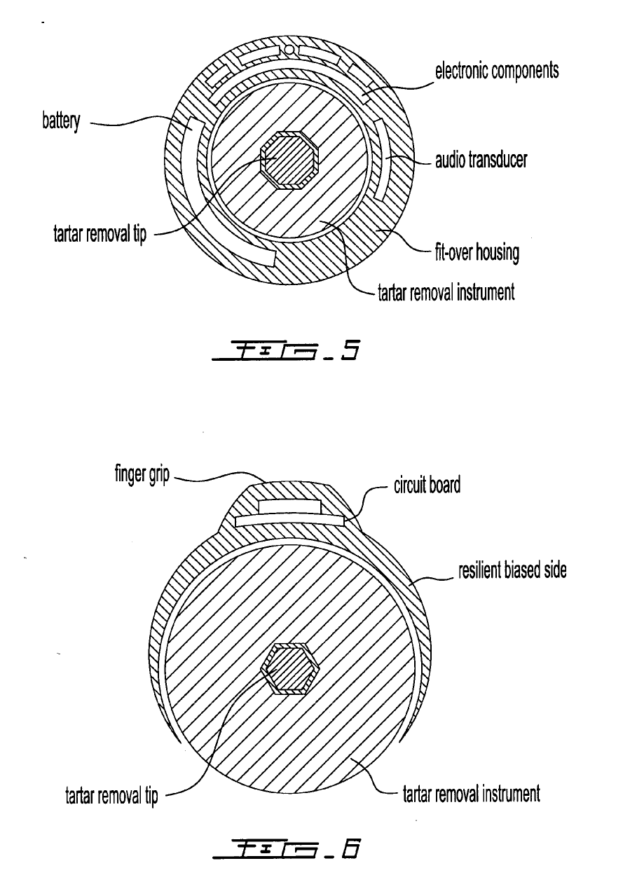

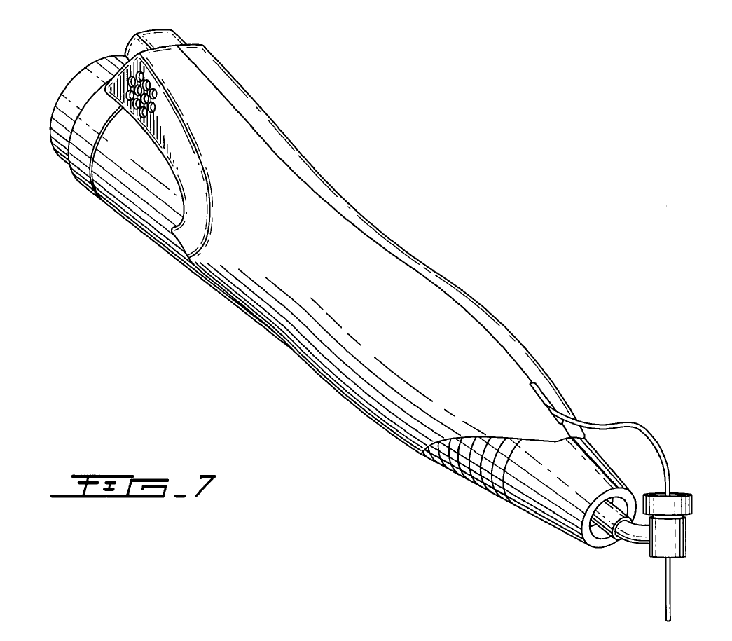

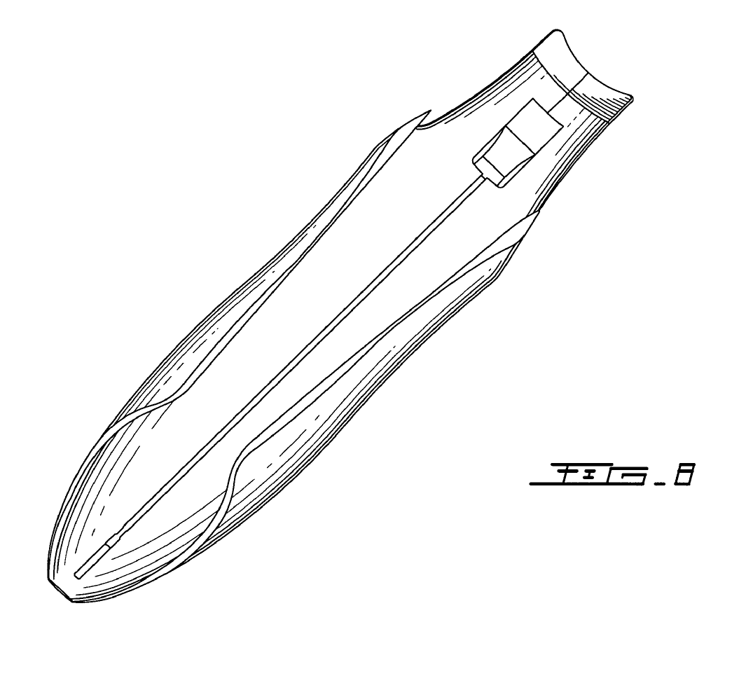

In the embodiment of FIGS. 6, 7 and 8, the optical tartar detection system has a housing that is adapted to slide over or clip to the tartar removal instrument. The housing may extend over the whole length of the instrument handle while a narrow elongated circuit board provides the electronic circuitry, optoelectronics and power source for the detection system. The side members are resiliently biased against the sides of the instrument. This embodiment allows for good gripping of the combination of the instrument and detection housing. The housing can be sterilized as needed. The optical fiber connection between the tip and the detection housing can be permanent, or preferably by way of an optical coupler so that the tip can be replaced separately without replacing the detection unit.

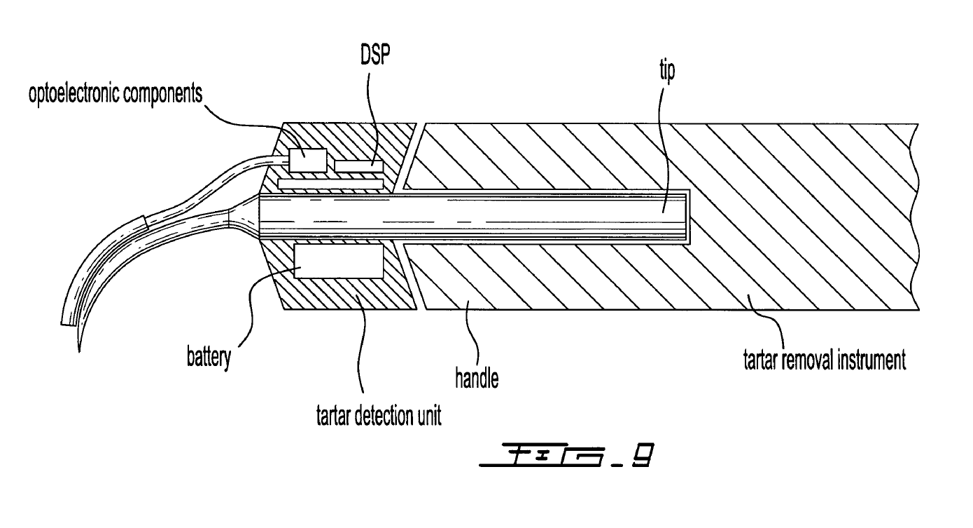

In the embodiment of FIG. 9, the optical tartar detection unit is self-contained and provided on a somewhat larger extension portion to a tartar removal tip as in the embodiment of FIG. 3. The usual optoelectronic components, electronic components, such as a DSP, and battery can be contained in a small annular housing on a shaft of the tip. As in the embodiment of FIG. 3, the optical light guide can be fed through a channel in the tip. The tip may also incorporate the structure of the embodiments of FIGS. 1a or 1b.

The technology can be used to perform the detection in reflectance, fluorescence, interferometry, Raman spectroscopy and the like.

The concept described herein could be used with another detection principle (having a connection means connecting to an analyzing mean) such as ultrasonic, sonic, acoustic and the like. The insert could also be designed to fit onto a laser.

The device of the invention may also comprise 3D global positioning, voice recognition and similar features that make its use more efficient.

While the invention has been described in connection with specific embodiments thereof, it will be understood that it is capable of further modifications and this application is intended to cover any variations, uses, or adaptations of the invention following, in general, the principles of the invention and including such departures from the present disclosures as come within known or customary practice within the art to which the invention pertains and as may be applied to the essential features herein before set forth, and as follows in the scope of the appended claims.

1-6. (canceled)

7. A sheath for covering a dental instrument tip, said sheath comprising a functional part and an optical light guide permitting the conduction of light for detection of dental pathologies.

8. The sheath as claimed in claim 7, wherein said tip is a scaling tip, said sheath is press-fit onto said tip, and said sheath is made of a ceramic material.

9. A dental pathology optical detection device for use in conjunction with a dental instrument having a handle and a functional part including a tip, said device comprising a housing adapted to removably attach to said handle by fitting around said handle, said housing containing a light source, a light detector, a signal processor for controlling said light source, receiving an output from said light detector and generating a detection signal, said housing providing a grip for a combined dental instrument and detection device.

10. The device as claimed in claim 9, wherein said dental instrument is a tartar removal instrument, and said detection device detects tartar.

11. The device as claimed in claim 9, wherein said housing is substantially ring-like and matches a cross-section of said handle.

12. The device as claimed in claim 9, wherein said housing comprises resilient sides that conform to and frictionally engage said handle.

13. The device as claimed in claim 9, wherein said housing is sterilizable.

14. A dental pathology optical detection device for use in conjunction with a dental instrument having a handle and a replaceable tip with a functional end and a connector end connected to said handle, said device comprising a connector end adapted to be connected to said handle, a functional end, a light source, a light detector, a signal processor for controlling said light source, receiving an output from said light detector and generating a detection signal.

15. The device as claimed in claim 14, wherein dental instrument is a tartar removal instrument, and said detection device detects tartar.

16. The device as claimed in claim 14, wherein said device comprises a sterilizable housing containing said light source, said light detector, and said signal processor, wherein said housing does not impede use of said handle when connected to said instrument.

17. (canceled)
